(19) United States
(12) Plant Patent Application Publication
(10) Pub. No.: US 2020/0337191 P1
(43) Pub. Date: Oct. 22, 2020

(54) Strawberry Plant Named 'SHANNON M. KENT'

(50) ;

(71) Applicant: Sweet Darling Sales, Inc., Aptos, CA (US)

(72) Inventor: John Larse, Watsonville, CA (US)

(21) Appl. No.: 16/873,474

(22) Filed: Apr. 17, 2020

Related U.S. Application Data
(60) Provisional application No. 62/835,705, filed on Apr. 18, 2019.

(51) Int. Cl. A01H 6/74

(52) U.S. Cl. PLT/208

(57) ABSTRACT

The present invention provides a new and distinct strawberry variety designated as ‘Shannon M. Kent’ (a.k.a. ‘110168’).

### CROSS-REFERENCE TO RELATED APPLICATIONS

This application claims priority to, and the benefit of U.S. Provisional Patent Application Ser. No. 62/835,705, filed Apr. 18, 2019, which is hereby incorporated by reference in its entirety for all purposes.

Latin name of the genus and species: Fragaria x ananassa.

Varietal denominations: ‘Shannon M. Kent’ (a.k.a. ‘110168’).

### BACKGROUND OF THE INVENTION

The present invention relates to a new and distinct strawberry variety designated as ‘Shannon M. Kent’ (a.k.a. ‘110168’), which was previously designated ‘Perla’, as disclosed in U.S. Provisional Patent Application No. 62/835,705.

‘Shannon M. Kent’ (a.k.a. ‘110168’) is the result of a controlled-cross between a female parent cultivar designated ‘107987’ and a male parent cultivar designated ‘107801’, and was first fruited in Watsonville, Calif. growing fields. Both parents are proprietary strawberry plant varieties made by the inventor and are not available to the public. Following selection and during testing, the plant was originally designated ‘110168’ and subsequently named ‘Shannon M. Kent’.

This new variety was asexually reproduced via runners (stolons) by the inventor at Watsonville, Calif. Asexual propagules from the original source have been tested in Watsonville growing fields and to a limited extent, grower fields in high elevation. The properties of this variety were found to be transmissible by such asexual reproduction. This cultivar is stable and reproduce true to type in successive generations of asexual reproduction.

### DESCRIPTION OF THE DRAWINGS

The accompanying color photographs depict various characteristics of the cultivar as nearly true as possible to make color reproductions.

FIG. 1 shows ‘Shannon M. Kent’ plant.

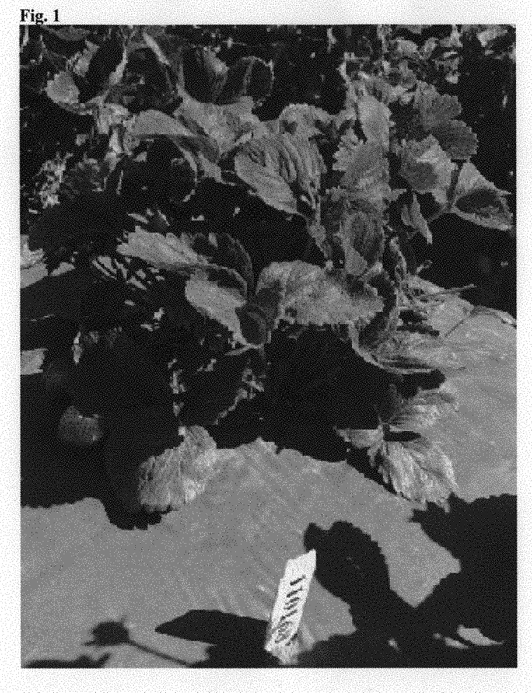

FIG. 2 shows ‘Shannon M. Kent’ flowers.

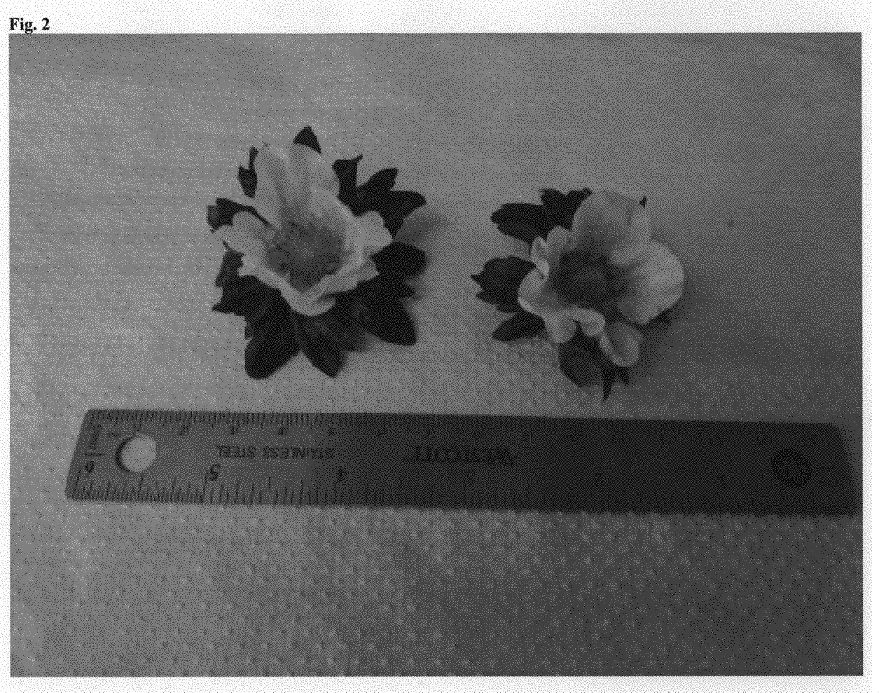

FIG. 3 shows ‘Shannon M. Kent’ leaf.

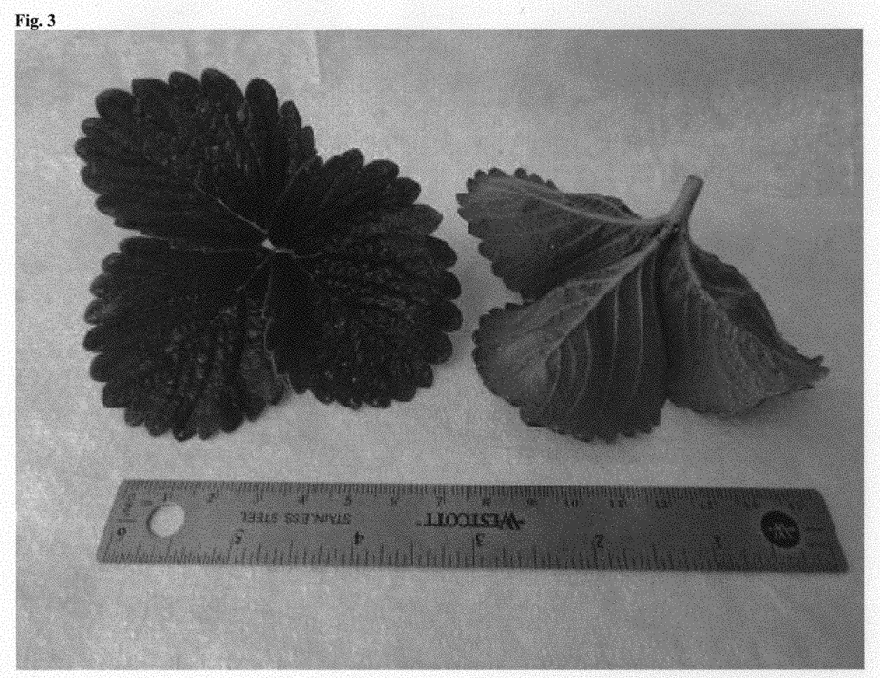

### SUMMARY OF THE INVENTION

This invention relates to a new and distinctive strawberry cultivar designated as ‘Shannon M. Kent’ (a.k.a. ‘110168’). This cultivar is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish the strawberry variety ‘Shannon M. Kent’ from known strawberry varieties. Plants for the botanical measurements in the present application were grown as annuals. Any color references are made to The Royal Horticultural Society Colour Chart, 1995 Edition, except where general terms of ordinary dictionary significance are used.

TABLE 1
Shannon M. Kent

| Character- | | |
|---|---|---|
| istic | | |
| Type | Characteristic | Observation |
| General | Plant Habit | annual |
| | Plant Growth Habit | semi-upright |
| | Day length | neutral |
| | Planting season | Fall |
| | Height | 30.5 cm |
| | Width | 38.5 cm |
| | Density of foliage | medium |
| | Plant vigor | moderate |
| | Freezing Quality | moderate |
| | Rain/weather tolerance | moderate |
| | Harvest Ease | moderate |
| Leaf | Leaf Shape | concave |
| | Terminal leaflet width | 76 mm |
| | Terminal leaflet length | 69 mm |
| | Terminal leaflet length/width ratio | 1.1 |
| | Teeth per terminal leaflet | 24 to 27 |
| | Shape of the terminal leaflet base | obtuse to rounded |
| | Shape of terminal leaflet in cross- | concave |
| | section | |
| | Shape of the terminal leaflet margin | serrate to crenate |
| | Color of upper side of leaflet | RHS color code 137A |
| | Color of lower side of leaflet | RHS color code 137C |
| | Leaf blistering | weak |
| | Leaf glossiness | medium |
| | Leaf variegation | absent |
| | Terminal Leaflet margin | flat to revolute |
| | Terminal Leaflet shape | Orbicular |
| | Terminal Leaflet shape of apex | Rounded |
| Limbs | Petiole length | 21.2 cm |
| | Petiole diameter | 3.65 mm |
| | Petiole pubescence | medium |
| | Petiole pose of hairs | slightly outwards |
| | Petiole color | RHS color code 145B |
| | Petiolule length | 0.4 to 0.7 cm |
| | Petiolule diameter | 2.00 to 3.33 mm |
| | Stipule length | 3.8 cm |
| | Stipule width | 1.0 cm |
| | Stipule pubescence | medium |
| | Stipule anthocyanin | present |
| | Stipule color (color code) | RHS color code 145A |
| | Pedicel color (color code) | RHS color code 145B |
| | Attitude of hairs on peduncle and | upwards |
| | pedicel | |
| Inflor- | Inflorescence position relative | above |
| escence | to foliage | |
| | Flower arrangement of petals | touching to overlapping |
| | Flower size | medium to large |
| | Flower diameter | 3.5 to 3.9 cm |
| | Petal shape | orbicular |
| | Petal apex | rounded |
| | Petal margin | entire |
| | Petal base shape | concave |
| | Petal length | 2.0 cm |
| | Petal width | 2.0 cm |
| | Petal length/ width ratio | 1 |
| | Petal number per flower | 6 |
| | Upper Petal color | RHS color code 155C |
| | Lower Petal color | RHS color code 155C |
| | Peduncle size | medium |
| | Floral Calyx Diameter | 5.0 to 5.6 cm |
| | Corolla diameter | 3.5 to 3.9 cm |
| | Calyx diameter relative to corolla | larger |
| | Inner calyx diameter relative to outer | equal to smaller |
| | calyx | |
| | Sepal shape | elliptical |
| | Sepal apex | convex |
| | Sepal margin | entire |
| | Sepal length | 2.6 cm |
| | Sepal width | 1.1 cm |
| | Sepal number per flower | 14 |
| | Fertility | not tested |
| | Time of flowering (50% of plants in | May |
| | bloom) | |
| | Shape of stigma | capitate |
| | Color of stigma | RHS color code 15B |
| | Length of style | 1.5 mm |
| | Color of style | RHS color code 6A |
| | Color of the ovary | RHS color code 145A |
| | Number of stamen | 25 to 30 |
| | Length of the stamens | 3.0 to 6.0 mm |
| | Shape of anther | dorsifixed |
| | Size of anther | 1.5 mm |
| | Color of anther | RHS color code 22A |
| | Color of filament | RHS color code 8C |
| | Length of filament | 2.5 to 5.5 mm |
| Stolon | Stolon number | 1 to 3 |
| Fruit | Fruit length | 4.15 cm |
| | Fruit width | 4.15 cm |
| | Fruit length/width ratio | 1 |
| | Fruit weight | 29.2 g |
| | Relative fruit size | medium to large |
| | Predominant fruit shape | globose conic |
| | Color of calyx | RHS color code 137A |
| | Pose of calyx segments | reflexed |
| | Firmness of flesh | firm |
| | Sweetness | 7.11 Brix |
| | Acidity | 3.49 pH |
| | Time of flowering | May |
| | Time of fruit ripening | May |
| | Harvest maturity (50% of plants with | June |
| | ripe fruit | |
| | Type of bearing | day netural |
| | Surface Texture | smooth |
| | Appearance Score | 3 |
| | Storage longevity | 10 days |
| | Cull rate: 1- % Usable | 14.5% |

‘Seascape’ (U.S. Plant Pat. No. 07,614) owned by The University of California is a commercial strawberry variety that is similar to, but distinguished from ‘Shannon M. Kent’. The fruit yield ‘Shannon M. Kent’ is greater than the fruit yield of comparison variety ‘Seascape’ and ‘Shannon M. Kent’ has a greater percent of marketable fruit than ‘Seascape’. The fruit height to fruit width ratio of the fruit of ‘Shannon M. Kent’ closer to unity compared to the fruit height to fruit width ratio of the fruit of ‘Seascape’. The petiole of ‘Shannon M. Kent’ is thicker in diameter than the petiole of ‘Seascape’. The florescence of ‘Shannon M. Kent’ extends beyond the leaf canopy and ‘Shannon M. Kent’ is easier to harvest compared to ‘Seascape’ with its habit of setting fruit beneath the canopy.

The new strawberry variety ‘Shannon M. Kent’ exceeds both its parents in fruit yield and the percent of marketable fruit of ‘Shannon M. Kent’ is less than the percent marketable fruit of female parent ‘107987’ and greater than that of male parent ‘107801’. The full fruiting season fruit yield of ‘Shannon M. Kent’ is greater than the yield of male parent ‘107801’, however, the male parent produced more fruit during the early season when compared to ‘Shannon M. Kent’. The fruit of ‘Shannon M. Kent’ is firmer than male parent ‘107801’. The fruit colour of ‘Shannon M. Kent’ is deeper red than the fruit colour of either of its two parents.

1. A new and distinct cultivar of strawberry plant named ‘Shannon M. Kent’ substantially as shown and described herein.
